(19) United States
(12) Patent Application Publication
(10) Pub. No.: US 2016/0108013 A1
(43) Pub. Date: Apr. 21, 2016

(54) PROCESSES FOR SELECTIVE EXTRACTION OF UNSAPONIFIABLE MATERIALS FROM RENEWABLE RAW MATERIALS BY REACTIVE TRITURATION IN THE PRESENCE OF A COSOLVENT

(71) Applicant: Saeml Valagro Carbone Renouvelable Poitou-Charentes, Poitiers Cedex (FR)

(72) Inventor: Antoine PICCIRILLI, Poitiers (FR)

(21) Appl. No.: 14/895,865

(22) PCT Filed: Jun. 4, 2014
(86) PCT No.: PCT/FR2014/051328

§ 371 Date: Dec. 3, 2015

(30) Foreign Application Priority Data
Jun. 4, 2013 (FR) 1355140

(51) Int. Cl. C07D 307/36

(52) U.S. Cl. CPC C07D 307/36

(57) ABSTRACT

A method for extracting an unsaponifiable fraction from a renewable raw material, includes the reactive trituration of the raw material dehydrated in the presence of at least one polar organic solvent including at least one light alcohol, at least one non-polar cosolvent immiscible with the light alcohol and at least one catalyst, resulting in the formation of a polar organic phase enriched with lipids functionalized with one or more function(s) chosen from hydroxyl, epoxide, ketone, thiol, aldehyde, ether and amine functions, and of a non-polar organic phase enriched with lipids containing no or few hydroxyl, epoxide, ketone, thiol, aldehyde, ether and amine function(s), and thereafter the concentration of the organic phases.

The present invention relates to the oleochemical field. More particularly, this invention relates to a method for extracting unsaponifiable matters from a lipidic renewable raw material, especially from an oleiferous fruit, in particular avocado, from an oleaginous seed or from a raw material derived from animals, algae, fungi or yeasts, or from a microorganism.

As used herein, lipids are intended to mean substances of biological origin that are soluble in non-polar solvents. Lipids may be saponifiable (for example triglycerides) or not saponifiable (for example molecules structured with a steroid-type skeleton).

As used herein, unsaponifiable matters are intended to include all the compounds, which, after complete saponification of a fat, that is to say under the sustained action of an alkaline base, remain insoluble in water and may be extracted by an organic solvent in which they are soluble. Unsaponifiable matters generally represent a minor fraction in fat.

There are five major groups of substances in most of unsaponifiable matters derived from vegetable fats: saturated or unsaturated hydrocarbons, aliphatic or terpene alcohols, sterols, tocopherols and tocotrienols, and carotenoid pigments, especially xanthophylls.

Lipidic renewable raw materials comprise highly variable proportions of unsaponifiable compounds. The unsaponifiable fraction contents obtained by extracting various vegetable oils according to different known methods range from 1 to 7% by weight of unsaponifiable matters in avocado oil, as opposed to 0.5% in coconut oil and 1% in soya or olive oil.

Currently, the traditional methods for extracting unsaponifiable matters generally use as a lipidic raw material vegetable oils and derivatives thereof and co-products from the lipid extraction industry (vegetable oils, animal fats, marine fats and oils, vegetable oleoresins), resulting from their refining and processing. Most of the time, it is necessary to extract the unsaponifiable matters from raw, semi-refined or refined vegetable oils, from unsaponifiable matter concentrates derived from refined oils obtained through a molecular distillation or through an extraction using supercritical fluids. Also, a number of unsaponifiable fractions such as sterols, squalene, tocopherols or tocotrienols are obtained from the vegetable oils from deodorization emissions, which are abundant co-products resulting from the chemical or physical refining of vegetable oils. However, to be mentioned as other co-products resulting from the refining of lipids are also acid-containing oils, soap pastes, lipids retained by bleaching earths that are used for decolorizing oils, earths retrieved from winterization units. Moreover, co-products resulting from oilseed or oleiferous fruit grinding may also be used, such as oil-cakes, seed husks or stones, molasses, black liquors.

In order to extract unsaponifiable matters or fractions thereof, co-products from the processing of lipids may also be used, such as raw glycerins from biodiesel production plants, resulting from animal or vegetable fat hydrolysis or saponification processes, greasy waters from animal fat processing industries, fatty acid alkyl ester still bottoms.

Likewise, unsaponifiable fractions are produced, especially sterols, from industrial co-products such as pulp production tall oil. Also to be mentioned are unsaponifiable fractions of co-products resulting from the extraction process of beverages, such as industrial breweries, rum distilleries, and malting plants.

As a raw material, source for unsaponifiable matters, can be further employed plant serums (ex. from tomatoes, citrus fruits), seeds, testae, oleoresins from fruits, that are oleiferous or not, from vegetables, flowers or leaves.

The methods for extracting unsaponifiable matters most of the time comprise a step of transesterification or esterification of the fat obtained by pressing, and/or a step of saponification of the fat, followed with a liquid-liquid extraction by means of an organic solvent.

The methods for selectively extracting unsaponifiable fractions are not numerous.

The application WO 2011/048339 describes a method for extracting an unsaponifiable fraction from a renewable raw material, comprising a) the dehydration and conditioning of the renewable raw material, b) the transesterification by an active trituration of the conditioned lipid raw material in the presence of a light alcohol and a catalyst, c) the evaporation of the light alcohol, d) the concentration of the liquid phase so as to obtain a concentrate comprising the unsaponifiable fraction diluted in fatty acid alkyl esters, e) the saponification of the unsaponifiable concentrate, f) the extraction of the unsaponifiable fraction from the saponified mixture.

Avocado, because of its unsaponifiable fraction high content should be considered with a very special attention. It allows in a known way the access to particular lipids of the furanic type, which major component is a linoleic furan noted H7 having the following formula:

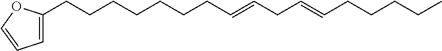 [SMILES: CCCCC/C=C/C/C=C/CCCCCCCc1ccco1]

As used herein, avocado-derived furan lipids are intended to mean components having the following formula:

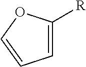

wherein R is a C11-C19, preferably a C13-C17, linear hydrocarbon chain, saturated or comprising one or more ethylene or acetylene unsaturations. These furan lipids from avocado have been described especially in Farines, M. and al, 1995, J. Am. Oil Chem. Soc. 72, 473. As a rule, furan lipids from avocado are compounds that are unique in the vegetable kingdom and are very particularly sought after for their pharmacological, cosmetic, and nutritional properties, or even as biopesticides.

Furan lipids from avocado are metabolites of precursor compounds that are initially present in the fruit and the leaves, and which, due to the effect of heat do dehydrate and cyclize to furan derivatives. As an example, linoleic furan H7 results from the heat transformation of following keto-hydroxyl precursor, noted P1H7:

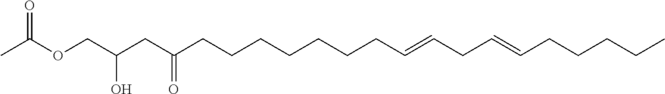 [SMILES: CCCCC/C=C/C/C=C/CCCCCCCC(=O)CC(O)COC(C)=O]

Under atmospheric pressure, precursor P1H7 is typically converted to linoleic furan H7 at a temperature ranging from 80 to 120° C.

It is today well established that the presence of these furanic compound precursors in the leaves or in the fruit of avocado (including the stone) not only depends on the variety (Hass and Fuerte varieties being the richest in such compounds) but also on the method for producing the oil or other vegetable extract of avocado (hexane or ethanol extract from avocado leaves).

Furthermore, some compounds that are initially present in avocado fruit and leaves may present in the form of polyhydroxylated fatty alcohols, most of the time non acetylated, such as the following compound:

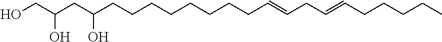 [SMILES: CCCCC/C=C/C/C=C/CCCCCCCCC(O)CC(O)CO]

As used herein, a polyhydroxylated fatty alcohol from avocado is intended to mean a polyol in the form of a C17-C21 straight main hydrocarbon chain, saturated or comprising one or more ethylene or acetylene unsaturations, and comprising at least two hydroxyl groups, said hydroxyl groups being generally located on one portion of the main chain, preferably in the direction of either of both ends thereof, the other portion of this main chain thus forming the fatty chain (hydrophobic portion) of the polyol.

The polyhydroxylated fatty alcohol content in the fruit mainly depends on the weather conditions, on the soil quality, on the season and on the ripening of the fruits when picked.

Considering the therapeutic interest of the avocado unsaponifiable, that is rich in furan lipids, for its beneficial and curative effect onto conjunctive tissues, especially against inflammatory diseases such as arthrosis, parodontitis and scleroderma, and further considering its generally high cost, there is a strong need for preparing with the best yield as possible, unsaponifiable fractions from avocado oil, that would be rich in furan lipids. Likewise, there is a real interest in positively using, with a maximum yield, the fruit as a whole, so as to improve the global cost effectiveness of the process.

The known methods to produce these furanic compounds or specific polyols from the fruit or from the oil extracted from the fruit avocado do only enable to obtain these compounds when combined with many other avocado-derived unsaponifiable compounds.

The French application FR 2678632 describes a method for producing the avocado unsaponifiable fraction from an avocado oil enriched with one of its fractions, called H, in fact corresponding to the same furan lipids. The preparation of such a furan lipid-rich unsaponifiable matter, which content may vary from 30 to 60%, essentially depends on the controlled heating of the fresh fruits, that have been beforehand thinly sliced, at a temperature ranging from 80 to 120° C., and for a period of time preferably chosen between 24 and 48 hours. This heat treatment enables after extraction, to obtain a furan lipid-rich avocado oil. Lastly, starting from this oil, the unsaponifiable fraction is obtained according to a traditional saponification method, completed with a step of liquid-liquid extraction using an organic solvent.

The application WO 01/21605 describes a method for extracting furan lipid compounds and polyhydroxylated fatty alcohols from avocado, comprising a heat treatment of the fruit at a temperature of at least 80° C. (controlled drying), the extraction of oil by cold pressing, the enrichment with unsaponifiable matter through cold crystallization or liquid-liquid extraction or molecular distillation, ethanolic potash-mediated saponification, unsaponifiable extraction in counter-current column with an organic solvent, followed with steps of filtration, washing, desolventizing, deodorization and final molecular distillation. This method makes it possible to obtain either a distillate comprising primarily avocado furan lipids, or a distillate comprising primarily avocado furan lipids and polyhydroxylated fatty alcohols. However such method only enables to take advantage of a minor part of the fruit.

Indeed, in this type of process, that oil forming the bottoms resulting from the step of concentration of the unsaponifiable matter by molecular distillation, i.e. around 90% of the oil extracted from the fruit, can hardly be positively reused. This strongly colored oil did indeed undergo a heat treatment through high temperature-distillation, which leads to an automatic and non-reversible destruction of the chlorophyllous pigments, as well as phospholipids, with a very detrimental effect on the future refining of the distilled crude oil. Only a highly advanced refining of this oil in the best case scenario enables to give a relatively acceptable color back to it. Refining requires a high consumption of inputs (such as bleaching earths), of energy and still remains very brutal for unsaturated fatty acids (isomerization). Lastly, an exogenous antioxidant must be added for the preservation of this refined oil for a commercially acceptable period of time. As a consequence, the thus refined oil can absolutely not be reused for human nutrition or in specialist pharmaceutical applications.

A further drawback of this method consists in the production of an oil cake unsuitable for animal feeding. The latter indeed contains antinutritional compounds (toxic H precursors, used as biopesticides, furan lipids) and proteins that have been highly degraded during the extraction by mechanical pressing of the air-dried fruits (de facto highly oxidized), which suffer from a very low digestibility. As a consequence, the oil cake or proteins thereof, cannot be used in animal feeding and even less in human nutrition, even if the flesh of the fruit is commonly consumed by humans (guacamole, fruit to be directly consumed).

In the same way, the noble polysaccharides within the fruit, such as perseitol and nanoheptulose, unique sugars in the vegetable kingdom, with demonstrated pharmaceutical, cosmetic and nutritional properties (for ex. improved liver function), are partially destroyed through a Maillard reaction and/or caramelization process induced by the mechanical pressure of the dehydrated fruits, or are made very difficult to extract because of the excessive interaction with the fiber and protein-containing matrix.

As a conclusion, this type of method only enables a poor reuse of the fruit, which can be estimated to be lower than 15%.

As a consequence, it remains necessary to improve the yield as well as the selectivity of the methods for extracting furan lipids and/or polyhydroxylated fatty alcohols from avocado.

There is thus still a need for a method for selectively extracting unsaponifiable matters from fat while preserving the fruit integrity for a better future reuse, which implementation would be economic and would make it possible to also recover co-products of glycerides with a higher added value than free fatty acids, or proteins and polysaccharides with a good nutritional quality. It further would be desirable to develop a method for high-yield extracting unsaponifiable matters relative to the polarity of their fractions. It is indeed desirable to provide a robust method to selectively produce the expected fractions without being detrimental to the other interesting fractions or parts of the fruit.

In response, it is an object of the present invention to provide a method for extracting an unsaponifiable fraction from a renewable raw material comprising lipids functionalized with one or more function(s) chosen from hydroxyl, epoxide, ketone, thiol, aldehyde, ether and amine functions, comprising the following steps:

a) dehydration, optionally preceded or followed with a conditioning of the renewable raw material,

b) reactive trituration of the lipid raw material dehydrated and optionally conditioned, in the presence of at least one polar organic solvent comprising at least one light alcohol, at least one non-polar cosolvent immiscible with said light alcohol and at least one catalyst, resulting in the formation of a polar organic phase enriched with lipids having been functionalized with one or more function(s) chosen from hydroxyl, epoxide, ketone, thiol, aldehyde, ether and amine functions,

c) concentration of the polar organic phase to obtain a mixture enriched with the unsaponifiable fraction, optionally preceded, accompanied or followed with a heat treatment at a temperature higher than or equal to 75° C., preferably higher than or equal to 80° C.,

and comprising optionally the following steps:

d) saponification of the mixture enriched with the unsaponifiable fraction,

e) extraction of the unsaponifiable fraction from the saponified mixture.

The present invention further relates to a method for extracting an unsaponifiable fraction from a renewable raw material, comprising the following steps:

a) dehydration optionally preceded or followed with a conditioning of the renewable raw material,

b) reactive trituration of the lipid raw material, dehydrated and optionally conditioned, in the presence of at least one polar organic solvent comprising at least one light alcohol, at least one non-polar cosolvent immiscible with said light alcohol and at least one catalyst, resulting in the formation of a non-polar organic phase enriched with lipids containing no or few hydroxyl, epoxide, ketone, thiol, aldehyde, ether and amine function(s),

c) concentration of the non-polar organic phase to obtain a mixture enriched with an unsaponifiable fraction,

and comprising optionally the following steps:

d) saponification of the mixture enriched with the unsaponifiable fraction,

e) extraction of the unsaponifiable fraction from the saponified mixture,

wherein said renewable raw material undergoes optionally a heat treatment at a temperature higher than or equal to 75° C., preferably higher than or equal to 80° C., before or during step b), preferably before step a), during step a) or between step a) and step b).

Both methods of the invention do differ in that the first method aims at recovering an unsaponifiable fraction soluble in a polar alcohol phase (or which precursors are soluble in such a phase), whereas the second method aims at recovering the unsaponifiable fraction soluble in a non-polar organic phase (or which metabolites are soluble in such a phase). In the case of an avocado, both methods, although different in numerous steps, are however both equally useful since they make it possible to selectively recover furan lipids from the unsaponifiable fraction with a high yield, while enabling the production of very high quality-coproducts, which can be positively reused: distilled alkyl esters of avocado oil, perfectly traced avocado glycerin, oil cakes with antinutritional compounds removed therefrom, which can be potentially used as sources of proteins, of oligopeptides, of perseitol and nanoheptulose, avocado fibers.

In the particular case of the avocado, the raw materials in the first method especially are not initially heated at a high temperature (they are only heated after the reactive trituration step), while they are heated before the reactive trituration step in the second method, so as to produce earlier the furanic compound characteristics of a thermally treated avocado. In the case of the first method, the reactive trituration step is implemented with avocados, which did not undergo such a heat treatment and thus, at this stage, do contain furan lipid precursors.

The present invention therefore aims at extracting an unsaponifiable fraction from a renewable lipid raw material, generally originating from a plant or an animal, preferably from a plant. This raw material may especially be chosen from oleiferous fruits, oleaginous seeds, oleoproteaginous seeds, seed hulls, oleaginous almonds, sprouts, fruit stones and cuticles, animal raw materials derived from algae, fungi or yeasts, or from a microorganism, and that are rich in lipids.

In a first embodiment, the implemented raw material is an oleiferous fruit, which may be, without limitation, olive, shea, amaranth, palm, buritti, tucuman, squash, Serenoa repens, African palm or avocado.

In a second embodiment, the raw material is a seed, a pit, a sprout, a cuticle or a stone from a vegetable raw material chosen from rapeseed, soybean, sunflower, cotton, wheat, corn, rice, grapes (seeds), walnut, hazelnut, jojoba, lupine, camelina, flax, coconut, safflower, crambe, copra, peanuts, jatropha, castor bean, neem, canker, Cuphea, lesquerella, Inca inchi, perilla, echium, evening primrose, borage, black currant, pine of Korea, China wood, cotton, poppy (seeds), sesame, amaranth, coffee, oats, tomatoes, mastic tree, marigold, karanja, rice bran, Brazil nuts, andiroba, schizandra, ucuhuba, cupuacu, murumuru, pequi, seeds from lemon oil, mandarin, orange, watermelon, Cucurbita pepo and tomato. The lipid raw material may also be a raw material derived from animals, algae, fungi or yeasts. To be mentioned as preferred animal raw materials are fish liver and skin, very especially those of shark, cod and chimera, as well as solid waste from the meat industry (brains, tendons, lanolin . . . ).

Other vegetable raw materials containing oleoresins that are rich in unsaponifiable matters are tomato, marigold, paprika, rosemary.

To be mentioned as suitable examples of algae containing interesting unsaponifiable compounds are microalgae Duniella salina (rich in beta-carotene) and Hematococcus pluvialis (rich in asthaxanthin). Suitable examples of microorganisms, especially bacteria containing interesting unsaponifiable compounds include mycelia or other mold and fungus (production of ergosterol), Phaffia sp. (producing asthaxanthin), Blakeslea trispora, (producing lycopene and phytoene), Muriellopsis sp. (producing lutein), or are especially mentioned in the application WO 2012/159980 (microalgae strain adapted to produce squalene), in the U.S. Pat. No. 7,659,097 (bacteria producing especially farnesol and farnesene), in the publication Pure & Appl. Chem., Vol. 69, No. 10, pp. 2169-2173, 1997 (production of carotenoids) or in Journal of Biomedicine and Biotechnology, 2012; 2012:607329, doi: 10.1155/2012/607329 (biotechnological production of co-enzyme Q10).

It is desirable that the raw materials used in the method of the invention have an acidity lower than 3 mg KOH/g. Indeed, higher contents in free fatty acids in these raw materials would cause the formation of soaps in a basic medium. As used herein, fatty acids are intended to mean C4-C28 mono-, di- or tricarboxylic aliphatic acids, saturated, monounsaturated or polyunsaturated, linear or branched, cyclic or acyclic, that may comprise some particular organic functions (hydroxyl, epoxy functions, . . . ).

The first method of the invention will now be presented in detail.

The raw materials that are implemented in the first method of the invention comprise lipid components functionalized with one or more polar function(s), chosen from hydroxyl, epoxide, ketone, thiol, aldehyde, ether and amine functions, as for example avocado, karanja, jatropha, andiroba, neem, schizandra, lupine hull, cashew nut, sesame, rice bran, cotton, or oil-producing raw materials that are rich in phytosterols such as corn, soya, sunflower, rapeseed, which all are very rich in such compounds.

These raw materials may be fresh raw materials or raw materials having been previously submitted to some processes, such as for example a first step of raw material extraction, such as pressing or centrifugation. As regards avocado, to be mentioned are avocado milks obtained by pressing pulps, products resulting from the removal of pulps, that have been partly defatted through centrifugation, by-products generally present at the outputs of sieve-centrifuges, centrifugation pellets produced upon separation, avocado cakes, simultaneously produced when the fruits are cold-pressed (fresh or dried) or during the liquid-solid extraction of avocado oil from fresh or dried fruits, by means of an organic solvent, avocado stones and leaves.

This method comprises a first step a) of dehydration and optionally of conditioning of the renewable raw material. Dehydration and conditioning, when conducted at a temperature lower than or equal to 80° C., preferably lower than or equal to 75° C., are said to be controlled (this is required for avocado). Said temperature is preferably higher than or equal to −50° C. According to another embodiment (not applicable to avocado), temperature varies from 50 to 120° C., more preferably from 75 to 120° C. Dehydration may be conducted under inert atmosphere, especially in the case of raw materials containing delicate compounds that may oxidize when temperature increases. It is preferably conducted under atmospheric pressure.

In the case of avocado (which is intended to mean, as used in the present application, the fruit, the stone, the leaves of avocado or their mixtures), not to rise temperature above 75 or 80° C. prevents the conversion of furan lipid precursors to furan lipids.

Dehydration may be implemented before or after conditioning (if needed). Preferably, oleiferous fruits like avocado are dehydrated prior to being conditioned, whereas oleaginous seeds on the contrary are first conditioned prior to being dehydrated.

As used herein, dehydration is intended to include all the techniques known from the person skilled in the art, which enable the total or partial removal of water from the raw material. Amongst these techniques are to be mentioned, without limitation, fluidized bed drying, drying under a hot air current or under an inert atmosphere (ex. nitrogen), packed-bed drying, under atmospheric pressure or under vacuum, thick-layer drying or thin-layer drying, in a continuous belt dryer in a hot air dryer with rotary fans, or microwave drying, spray drying, freeze-drying and osmotic dehydration, in a solution (direct osmosis), or in a solid phase (ex. drying in osmotic bags), drying using solid absorbents, such as zeolites or molecular sieves.

More preferably, the drying time and temperature are chosen so that residual moisture be lower than or equal to 3% by weight, preferably lower than or equal to 2%, as compared to the weight of the lipid raw material obtained at the end of the dehydration step. The residual moisture of the raw material may be determined by thermogravimetry. This drying step is important so that the subsequent transesterification step proceeds under the best conditions. It will make the lipid component extraction more efficient, because it especially makes the cells of the raw material burst, and the oil-in-water emulsion break, such as present in this raw material. Moreover it may facilitate the conditioning of the raw material, especially the crushing or milling operations, which will make the solvent-mediated extraction more efficient because of the benefit in terms of contact surface with the solvents.

Within the frame of the present method, so as to facilitate an industrial implementation and for cost reasons, drying in thermoregulated, vented dryers (drying ovens), in thin layers and under a hot air current, is preferred. The temperature does preferably range from 70 to 75° C., and dehydration lasts preferably for 8 to 36 hours.

The aim of the -optional-conditioning of the raw material is to make the fats the most accessible to the extraction solvents and to catalysts, especially through a simple phenomenon of percolation. Conditioning may also increase the specific surface and porosity of the raw material in contact with these reagents. The conditioning of the raw material does not lead to any fat extraction.

Preferably, the renewable raw material is conditioned by flattening, flocking, blowing or grinding in the form of a powder. As an example, the raw material may be toasted or flocked, or conditioned and/or freeze-dried, dried through evaporation, spraying, mechanical grinding, freeze-grinding, dehulling, flash-relaxation (quick drying by creation of vacuum and quick depressurization), conditioned with pulsed electromagnetic fields, by reactive or non-reactive extrusion, flattening by means of a mechanical flattener with smooth rollers or corrugated rollers, blowing through hot air or superheated vapor supply. In the case of avocado, primarily cut avocado fruits will be used, which will be thereafter submitted to a controlled dehydration step, and lastly the dried fruit will be conditioned, generally by grinding the fresh pulp.

Once dehydrated and optionally conditioned, the raw material is submitted to a step b) of reactive trituration in the presence of at least one polar organic solvent comprising at least one light alcohol, of at least one non-polar cosolvent immiscible with said light alcohol (in the conditions of the reactive trituration operation) and of at least one catalyst.

As used herein, a reactive trituration is intended to mean any operation aiming at converting lipids (or fats), that are saponifiable (in particular triglycerides) to fatty acid alkyl esters (generally fatty acid alkyl monoesters) and to glycerol, preferably in the presence of one or more reagent(s). In the present case, trituration is effected in the presence of a light alcohol, of a non-polar cosolvent and a catalyst. In a particular embodiment, anhydrous solvents and cosolvents will be used, and preferably solvents with a sufficiently low boiling point to allow distillation.

In a further embodiment, water can be added to the binary mixture of solvents so as to extract especially more efficiently highly polar compounds, in particular hydroxylated compounds, wherein the amount of water preferably represents from 0.1 to 20% by weight of the mixture of solvents, preferably from 0.5 to 5%.

This step not only enables to extract fats, in particular oil, from the dehydrated raw material, while simultaneously transesterifying the same, but also to isolate a fraction enriched with polar lipid components, containing one or more function(s) chosen from hydroxyl (preferably aliphatic), epoxide, ketone, thiol, aldehyde, ether and (free) amine functions, whether unsaponifiable or not, as well as a fraction enriched with non-polar or weakly polar lipid components, especially components which do not contain any hydroxyl, epoxide, ketone, thiol, aldehyde, ether and amine functions.

The addition of a non-polar cosolvent promotes the formation of a heterogeneous medium and of two lipid phases, which will be very different from each other as regards their composition. On one hand, lipid components non functionalized with one or more hydroxyl, epoxide, ketone, thiol, aldehyde, ether and amine function(s) will be found preferably in the non-polar phase, whereas lipid components functionalized with one or more hydroxyl, epoxide, ketone, thiol, aldehyde, ether or amine function(s) will be found preferably in the polar phase (light alcohol).

This step enables the selective extraction of lipid components (unsaponifiable or not) functionalized with one or more hydroxyl (preferably aliphatic), epoxide, ketone, thiol, aldehyde, ether or amine function(s), preferably several of them, which are not separated from the lipid component mixture (especially fatty acid esters) not comprising such functions, present in the medium at the end of the transesterification reaction. Depending on the type of raw material used, these functionalized lipid components can be, without limitation, polyhydroxylated fatty alcohols and keto-hydroxylated compounds, furan lipid precursors (especially compound P1H7 previously mentioned, precursor of linoleic furan H7) which are present in avocado, non esterified sterols, or esters of the following fatty acids: ricinoleic acid (12-hydroxy cis 9-octadecenoic acid) especially present in castor oil, lesquerolic acid (14-hydroxy-11-eicosanoic acid), densipolic acid (12-hydroxy-9,15-octadecadienoic acid) and auricolic acid (14-hydroxy-11,17-eicosadienoic acid), all three especially present in species of the Lesquerrella genus, coriolic acid (13-hydroxy-9,11-octadecadienoic acid), kamlolenic acid (18-hydroxy-9,11,13-octadecathenoic acid), especially present in oil extracted from seeds of the Kamala tree, coronaric acid (9,10-epoxi-cis-octadec-12-enoic) especially present in sunflower oil, vernolic acid (cis-12,13-epoxioleic acid) especially present in oil extracted from seeds of Euphorbia lagascae or from plants of the Vernonia genus.

Step b) is conducted in temperature, stirring and time conditions sufficient to enable the extraction of triglycerides and other lipid components from the raw material and the transesterification of said triglycerides, leading to the formation of a mixture comprising especially fatty acid esters, glycerol, the native unsaponifiable fraction (unmodified by this step), and depending on the type of raw material used, soluble polysaccharides, phenolic compounds, glucosinolates, isocyanates, polar alkaloids, polar terpenes, glycerol and an oil cake.

Step b) however is conducted at a temperature lower than or equal to 80° C., preferably lower than or equal to 75° C. in the case of avocado especially, such temperature control preventing furan lipid precursors to be converted to furan lipids. These remain present in their hydroxylated form (not cyclized to furans) during the reactive trituration.

In other cases, step b) may be conducted without limitation as regards temperature, that is to say the temperature may be set over 75 or 80° C. Thus, when the raw material is not derived from avocado, step b) may be conducted by implementing a heating process at a temperature ranging from 40 to 100° C. Step b) generally is conducted at room temperature but may also be conducted by implementing a heating process, at a temperature preferably of at least 40° C. and preferably lower than or equal to 80° C., preferably lower than or equal to 75° C.

General publications such as Bailey's Industrial Oil and Fat Products, 6^{th} Edition (2005), Fereidoon Shahidi Ed., John Wiley & Sons, Inc., and March's Advanced Organic Chemistry: Reactions, Mechanisms, and Structure, 5^{th} Edition (2001), M. B. Smith, J. March, Wiley-Interscience, describe in more details the conditions of the transesterification step, as well as the optional saponification step, which will be presented thereafter.

As used herein, a light alcohol is intended to mean an alcohol (comprising one or more hydroxyl functions), which molecular weight is lower than or equal to 150 g/mol, linear or branched, preferably C₁-C₆, more preferably, C₁-C₄. Preferably the light alcohol is a monoalcohol. It is preferably an aliphatic alcohol and most preferably an aliphatic monoalcohol, preferably chosen from methanol, ethanol, n-propanol, isopropanol, n-butanol, n-pentanol, n-hexanol, ethyl-2-hexanol, and isomers thereof. Using such a monoalcohol, most preferably methanol, will lead to the conversion of glycerides to fatty acid monoesters.

The non-polar cosolvent, immiscible with the light alcohol (in the conditions of the reactive trituration), is preferably chosen so that lipid components, functionalized with one or more hydroxyl, epoxide, ketone, thiol, aldehyde, ether or amine function(s), to be extracted, are not soluble in this cosolvent. Considering their chemical nature, these functionalized lipid components will have necessarily a stronger affinity with the light alcohol phase than with the non-polar solvent phase, in which they are not much (preferably not) soluble.

The non-polar cosolvent is an organic solvent which may especially be hexane, heptane, benzene, bicyclohexyl, cyclohexane, paraffin alkanes of vegetable origin obtained by dehydration of natural alcohols (or their Guerbet homologues) or by hydrotreatment of lipids or biomass (hydroliquefaction method) or by decarboxylation of the fatty acids, decaline, decane, kerosine, kerdane (a combustible hydrocarbon cut heavier than hexane), gas oil, lamp oil, methylcyclohexane, tetradecane, supercritical CO₂, pressurized propane or butane, natural non-polar solvents such as terpenes (limonene, alpha- and beta-pinene, etc.). It will preferably be an alkane or a mixture of alkanes, preferably hexane.

The preferred polar solvent (light alcohol)/non-polar cosolvent couple is the methanol/hexane couple.

The catalyst is preferably a basic catalyst preferably chosen from alcoholic soda, solid soda, alcoholic potash, solid potash, alkaline alcoholates, such as lithium, sodium or potassium methylate, ethylate, n-propylate, isopropylate, n-butylate, i-butylate or t-butylate, amines and polyamines, or an acid catalyst preferably chosen from sulfuric acid, nitric acid, paratoluenesulfonic acid, hydrochloric acid and Lewis acids. An acid catalyst will be more particularly used in extreme situations, where free acidity of the fat will be higher than 4 mg KOH/g. This step will lead to the esterification of free fatty acids, and the continuation of the method consists, after the reactive trituration, in implementing a base-catalyzed transesterification reaction.

Step b) may be conducted especially in a batch reactor with a stirred bed or in a continuous reactor with a mobile belt, of the continuous extractor type. In a preferred embodiment, the organic solvent and the non-polar cosolvent are introduced in counter-current to each other into a reactor. To optimize the separation of the various lipid components between polar and non-polar phases, and/or to obtain a complete conversion of the mono-, di- and triglycerides to fatty acid (alkyl)(mono)esters, the extraction/trituration process may be repeated several times, for example by implementing several reactors in a cascade and intermediate draw-off systems, such as described in the application WO 2010/084276.

The reactive trituration step enables to recover (especially after filtration and washing of the oil cake with a solvent such as a light alcohol) on one hand two liquid, non miscible lipid phases, glycerol, and on the other hand a solvent-containing oil cake.

Most preferably, the mixture resulting from the transesterification step comprises small amounts of mono-, di- or triglycerides. The glycerides, as a whole, represent generally less than 3% by weight of the mixture total weight, preferably less than 1%.

The solvent-containing oil cake resulting from the method of the invention may be dried, then be directly used especially animal feeding, being given that it does not contain, or then at least very few of, antinutritional compounds following the reactive trituration step, as opposed to methods of the prior art which imply a step of mechanical pressure. The (alcoholic) polar phase in which are especially soluble lipids containing one or more functions chosen from hydroxyl, epoxide, ketone, thiol, aldehyde, ether and amine functions, such as polyhydroxylated fatty alcohols and furan lipid precursors (in the case of avocado) is separated from the non-polar phase. Said polar phase further comprises especially fatty acid esters. The separation of the various fractions may occur in many different ways, especially by centrifugation, decantation and/or distillation.

Thus the non-polar solvent phase may be submitted to a solvent evaporation step conducted under vacuum and at a suitable temperature. The vaporized solvent is then condensed for being recycled. The non-polar heavy phase (phase A), primarily formed of alkylesters and unsaponifiable (or not) non-polar compounds may then be engaged in the molecular distillation so as to obtain, on one hand, purified esters (in the distillate) and, on the other hand, a distillation residue enriched with non-polar minor compounds. The extraction of these essentially unsaponifiable compounds is conducted according to methods that are known to the person skilled in the art. For example by conducting the following sequence: 1) saponification of the alkylesters, 2) liquid-liquid extraction enabling to separate the unsaponifiable compounds from the soaps, 3) desolventizing of the solvent phase enriched with unsaponifiable matters and 4) final purification of the unsaponifiable matter.

Another alternative consists in directly saponifying phase A and in extracting the essentially non-polar unsaponifiable compounds by 1) a liquid-liquid extraction enabling to separate the unsaponifiable compounds from the soaps, 2) a desolventizing of the solvent phase enriched with unsaponifiable matters and 3) the final purification of the unsaponifiable matter.

The light alcohol (polar solvent) is evaporated from the polar phase especially under reduced pressure. In the case of avocado, if the evaporation temperature is high (especially of around 80° C. or above), a cyclization of the furan lipid precursors to furan lipids may already occur at this early stage.

The lipid product obtained may be submitted to a step of neutralization (before or after the evaporation of the light alcohol, preferably before), preferably through an acid, then a step of decantation or centrifugation which enables to recover glycerol residue on one hand and a lipid phase on the other hand, and/or a step of filtration. The remaining lipid phase may then be washed with water and dried under vacuum.

The resulting lipid phase (phase which typically contains alkylesters and enriched with polar unsaponifiable (or not) compounds) is then submitted to a step c) of concentration to obtain a mixture enriched with an unsaponifiable fraction and optionally to a heat treatment at a temperature higher than or equal to 75° C., preferably higher than or equal to 80° C. The concentration may be implemented before or after the heat treatment, if any, or these two steps may be conducted concomitantly, if the concentration requires a heating process at a suitable temperature. The concentration is preferably carried out prior to effecting the heat treatment.

The preliminary concentration of oil to unsaponifiable enables to reduce the amount of engaged matter upon the possible subsequent step of saponification, and thus the amount to be extracted.

The concentration step c) may in particular be conducted by liquid-liquid extraction, distillation or crystallization, especially cold crystallization or crystallization through evaporation under vacuum. As used herein, distillation is intended to mean any method known from the person skilled in the art especially, molecular distillation, distillation under atmospheric pressure or under vacuum, multi-stage, serially (especially in a wiped film evaporator or a falling film evaporator), azeotropic distillation, hydrodistillation, steam distillation, deodorization especially in thin-layer deodorizer under vacuum with or without steam injection or inert gas injection (nitrogen, carbon dioxide).

The most preferred method is the molecular distillation, which is intended to mean a fractional distillation under high vacuum and high temperature, but with a very short contact time, which prevents or limits the denaturation of heat-sensitive molecules.

This step of molecular distillation, as well as all other molecular distillations that can be carried out in the methods of the present invention, is conducted by using a short-path distillation unit, preferably a device chosen from molecular distillation devices of the centrifuge type and molecular devices of the wiped-film type.

Molecular distillation devices of the centrifuge type are known from the person skilled in the art. For example, the application EP-0 493 144 describes a molecular distillation device of this type. Generally speaking, the product to be distilled is spread in a thin layer on the heated surface (hot surface) of a conical rotor rotating at high speed. The distillation chamber is placed under vacuum. In these conditions, an evaporation of the unsaponifiable components occurs, not an ebullition, from the hot surface, the advantage being that delicate products are not degraded during evaporation.

Molecular distillation devices of the wiped-film type, also known from the person skilled in the art, comprise a distillation chamber provided with a rotating scraper, enabling the continuous spreading onto the evaporation surface (hot surface) of the product to be distilled. The vapors of product are condensed by means of a cold finger, placed in the middle of the distillation chamber. The external power and vacuum supply systems are very similar to those of a distillation unit of the centrifuge type (supply pumps, vacuum pumps with sliding vanes and oil diffusion, etc.). The recovery of residues and distillates in glass flasks occurs by gravitational flow.

The molecular distillation is conducted preferably at a temperature ranging from 100 to 260° C. by keeping a pressure ranging from 10⁻³ to 10⁻² mm Hg and preferably of about 10⁻³ mm Hg. These conditions are softer than those of the methods of the prior art which imply a distillation of triglycerides instead of fatty acid monoesters, which enables to avoid the decomposition of the colored pigments leading to a nearly non-reversible coloring of the residue.

The concentration of unsaponifiable matter in the distillate may reach 60% by weight. In the case of avocado, even if the contact time of the compounds with the heated area is very short, some furan lipid precursors may be cyclized to furan lipids at this stage. Such phenomenon however remains marginal. It is also possible to effect a classical distillation, which, In the case of avocado, would enable a complete cyclization of the furan lipid precursors through a heating process at 75° C. or above, preferably at 80° C. or above.

Distillation generally enables to obtain a light fraction (first distillate), comprising esters (typically alkylesters) of fatty acids of high purity, separated from the unsaponifiable fraction, and at least one heavier fraction (second distillate or residue), comprising the unsaponifiable fraction diluted in esters (typically alkylesters) of residual fatty acids.

The high purity fatty acid ester-containing fraction (that is to say generally clear and colorless esters) having preferably an ester content higher than 98% by weight and an unsaponifiable content preferably lower than 1%, more preferably lower than 0.1% by weight, may be directly used especially in cosmetics or in pharmacy. If the purity of the ester fraction obtained at the end of the concentration step is not sufficient, this fraction may be refined so as to improve the purity thereof, especially by molecular distillation.

In the case of avocado, the concentrate enriched with the unsaponifiable fraction (and depleted in fatty acid esters) contains at this stage furan lipid precursors (that are weakly volatile) and/or furan lipids (that are less volatile than the fatty acid monoesters), if the heat treatment step, which will be described hereafter, is conducted before or during the concentration step.

In the case of avocado, the heat treatment step at 75-80° C., or above, of the lipid phase having been concentrated, or not, is compulsory. It is intended to make the cyclization of the furan lipid precursors to furan lipids effective. This step may be conducted before or after the saponification step (if any), preferably before, because saponification would otherwise convert the furan lipid precursors to modified unsaponifiable derivatives (that is to say different from the furanic compounds), which would be less interesting. The duration of such treatment generally ranges from 0.5 to 5 hours, depending on the heating method used. The temperature set for the treatment is generally lower than or equal to 150° C., preferably lower than or equal to 120° C. It should be naturally understood that temperature and reaction time are two parameters that strongly depends from each other as regards the expected result of the heat treatment, which consists in promoting the cyclization of the furan lipid precursors.

Advantageously, this heat treatment is carried out under inert atmosphere, especially under nitrogen continuous flow. It is preferably conducted under atmospheric pressure.

The heat treatment step may be implemented in the presence, or not, of an acid catalyst. As used herein, an acid catalyst is intended to mean mineral and organic catalysts, said to be homogeneous, such as hydrochloric, sulfuric, acetic or paratoluenesulfonic acids, but also, and preferably, heterogeneous solid catalysts, such as silica, alumina, silica-alumina, zirconias, zeolites, acidic resins. Acidic aluminas with high specific areas will be in particular selected, that is to say at least equal to 200 m²/g. Preferred for implementation of the method of the invention are catalysts of the acidic alumina type.

The concentrate having optionally undergone the heat treatment may then be submitted to steps of d) saponification of the mixture enriched with the unsaponifiable fraction and e) extraction of the unsaponifiable fraction from the saponified mixture, depending on the type of raw material used. In the case of avocado, especially, steps d) and e) are effected, so as to separate glycerides. In other cases, steps d) and e) can be omitted and oil containing the unsaponifiable fraction, together with other compounds, can be isolated, such as (mono)fatty acid esters.

Saponification is a chemical reaction, which converts an ester to a water-soluble carboxylate ion plus alcohol. In the present case, saponification especially transforms fatty acid esters to fatty acids plus alcohol, the released alcohol being primarily the light alcohol used during the reactive trituration step to make transesterification effective.

The saponification step may be implemented in the presence of potash or soda in an alcoholic medium, preferably ethanol. Typical experimental conditions include a reaction in the presence of potash 12N under reflux of ethanol for 4 hours. At this stage, and optionally, a cosolvent may be advantageously used so as to improve in particular the reaction kinetics or to protect unsaponifiable compounds sensitive to basic pH values. This cosolvent may especially be chosen from terpenes (limonene, alpha- and beta-pinene, etc.), alkanes, especially paraffins.

Thereafter the unsaponifiable fraction is one or more times extracted from the saponified mixture. This step is preferably effected by liquid-liquid extraction by means of at least one suitable organic solvent, that is to say, which is immiscible with the alcoholic or hydroalcoholic solution resulting from the saponification. It enables to separate the fatty acid salts (soaps) formed during the saponification process of the unsaponifiable fraction.

The organic solvent may especially be a synthetic organic solvent chosen from optionally halogenated alkanes (especially petroleum ether or dichloromethane), aromatic solvents (especially trifluorotoluene, hexafluorobenzene), halogeno-alkanes, ethers (especially diethyl ether, diisopropyl ether, methyltertiobutyl ether, methyl tetrahydrofuran, 2-ethoxy-2-methylpropane), ketones (especially methyl isobutyl ketone, 2-heptanone), propionates (especially ethyl propionate, n-butyl propionate, isoamyl propionate), hexamethyldisiloxane, tetramethylsilane, diacetone alcohol, 1-butoxymethoxy butane, 3-methoxy-3-methyl-1-butanol (MMB), or a natural organic solvent chosen from terpenes, such as limonene, alpha pinene, beta pinene, myrcene, linalol, citronellol, geraniol, menthol, citral, citronellol, or oxygenated organic derivatives of natural origin especially ethers, aldehydes, alcohols and esters, such as for example furfural and furfurol. A terpene will be preferably chosen. The extraction may be conducted in a co- or counter-current extraction column or by means of a battery of mixer-settlers, extraction columns or centrifugal extractors.

In order to be adapted to the industrial scale, a continuous extraction can be provided in a device for a continuous liquid-liquid extraction, such as in a pulsed column, a mixer-settler or equivalents.

Once extracted, the unsaponifiable fraction is preferably purified, in particular by centrifugation (soap removal), desolventizing, washing, drying, filtration and/or deodorization under vacuum. More precisely, the purification step may especially be conducted by implementing one or more of the following sub-steps:
centrifugation of the solvent phase so as to extract the residual soaps, then filtration,
washing with water optionally saturated with sodium chloride of the solvent phase, in order to remove the alkaline residual traces,
drying through evaporation of the extraction solvent through distillation under vacuum, hydrodistillation or azeotropic distillation,
deodorization under vacuum of the unsaponifiable fraction so as to extract therefrom, in the deodorization conditions, any remaining contaminant especially the extraction solvent, pesticides, polycyclic aromatic hydrocarbons.

The first method of the invention enables to obtain a high-purity unsaponifiable fraction enriched with polar compounds (except, this is particular, in the case of avocado, furan lipids, which due to their weakly polar nature, are present in the unsaponifiable fraction isolated with the first method of the invention, because they have been formed in situ from polar precursors after a selective extraction step of the polar compounds). In a non-exhaustive manner, the unsaponifiable compounds obtained at the end of the implementation of the present method in the fraction isolated in fine may be, depending on the nature of the raw material used, optionally polyhydroxylated fatty alcohols, furan lipids (in the case of avocado), sterols and non-esterified (free) or non-glycosylated triterpene alcohols, free and glycosylated polyphenols, free or sulfated cholesterol, lignanes, phorbol esters, triterpenic acids (for ex. ursolic acid), polar terpenes (mono-, di- and sesqui-terpenes, with an alcohol function), alkaloids, polycosanols, limonoids, xanthophylls (lutein, astaxanthin, zeaxanthin) in a free form, gossypol, karanjin, shizandrin, azadirachtin, co-enzyme Q10, aflatoxins, especially B1 and B2, isoflavones, caffeine, theobromine, yohimbine, sylimarin, lupeol, althetoin.

In a general way, the average composition of an avocado unsaponifiable obtained following these different steps (amongst which steps d) and e)) as expressed in percentages by weight compared to the unsaponifiable total weight is as follows:
furan lipids 50-75%
polyhydroxylated fatty alcohols 5-30%
squalene 0.1-5%
sterols 0.1-5%
others 0-15%

According to the present invention, the unsaponifiable matter obtained as described may then be submitted to a (second) step of distillation, so as to further improve the purity thereof, preferably a molecular distillation, conducted preferably at a temperature ranging from 100 to 160° C., more preferably from 100 to 140° C., under a pressure ranging preferably from 10⁻³ to 5.10⁻² mm Hg. According to another embodiment, the set temperature varies from 130 to 160° C.

The temperature and pressure chosen for this distillation influence the formation of the recovered distillate. Thus, this (second) distillation may enable to obtain a distillate comprising primarily, in the case of avocado, avocado furan lipids, the purity of which may be higher than 90% by weight, when the distillation temperature varies from 100 to 140° C. When the distillation temperature varies from 130 to 160° C., a distillate is generally obtained comprising primarily avocado furan lipids and to a lesser extent polyhydroxylated fatty alcohols from avocado, which combined amounts may exceed 90% by weight.

This first method of the invention enables thus to provide a selective extraction not only of the avocado furan lipids, but also of avocado polyhydroxylated fatty alcohols, if desired.

Furthermore, the unsaponifiable compounds obtained at the end of the implementation of the method in the fraction isolated from the non-polar solvent phase, may be in fine, depending on the nature of the raw material used, sterol esters, esterified triterpene alcohols, cholesterol esters, tocopherols (and corresponding tocotrienols), sesamolin, sesamin, sterenes, squalene, paraffin hydrocarbons, weakly to non-polar terpenes (mono-, di- and sesqui-terpenes with an aldehyde and/or a ketone function), esterified xanthophylls (lutein, astaxanthin, zeaxanthin), carotenoid type pigments (beta-carotene, lycopene), waxes, calciferol, cholecalciferol, pongamol.

The second method of the invention will now be presented by explaining essentially the differences as compared to the first method of the invention. It should be noted that the description of the first method of the invention can be referred to, as regards all other characteristics, which are common to both methods.

The renewable raw materials used in the second method of the invention are not particularly limited and optionally comprise lipid components functionalized with one or more hydroxyl, epoxide, ketone, thiol, aldehyde, ether or amine function(s). They comprise necessarily those lipid components, which are not functionalized by any of the previously mentioned functions (or by a few number of these functions), these components being the most commonly encountered in nature.

This method comprises a first step a) of dehydration, and optionally of conditioning, of the renewable raw material. Dehydration and conditioning are not necessarily conducted at a temperature lower than or equal to 80° C. or 75° C. Said temperature is preferably higher than or equal to −50° C. When a heating process is provided, the temperature generally varies from 50 to 120° C., more preferably from 75 to 120° C.

As for the first method, dehydration may be implemented before or after conditioning (if any). It lasts preferably from 8 to 36 hours.

The renewable raw material optionally undergoes (this is the case for avocado in particular) a heat treatment as described especially in the French patent application FR 2678632, at a temperature higher than or equal to 75° C., preferably higher than or equal to 80° C., before or during step b), preferably before step a), during step a) or between step a) and step b) of reactive trituration. Most preferably, the heat treatment and the dehydration of the raw material occur simultaneously and form a single step.

In the case of avocado, this heat treatment step at 75° C. or above of the raw material having been beforehand, or not, conditioned and/or dehydrated, is compulsory. As for the first method described, it is intended to promote the cyclization of the furan lipid precursors to furan lipids. The duration of such treatment generally varies from 8 to 36 hours, depending on the heating method used. The temperature set for the treatment is generally lower than or equal to 150° C., preferably lower than or equal to 120° C. Advantageously, a heat treatment is conducted under inert atmosphere, especially under a nitrogen continuous flow. It is preferably conducted under atmospheric pressure.

Once dehydrated and optionally conditioned, the raw material undergoes a step b) of reactive trituration in the presence of at least one polar organic solvent comprising at least one light alcohol, at least one non-polar cosolvent immiscible with said light alcohol, and at least one catalyst. As in the first method, these solvents and cosolvents may be anhydrous or not, and water may be added to the extraction solvent mixture.

This step enables on one hand to extract fats, in particular the oil from the dehydrated raw material and simultaneously to transesterify the same, and on the other hand to isolate a fraction enriched with lipid components, which does not contain (or few of them) any hydroxyl, epoxide, ketone, thiol, aldehyde, ether and amine function, and a fraction enriched with polar lipid components, especially functionalized with one or more hydroxyl (preferably aliphatic), epoxide, ketone, thiol, aldehyde, ether or amine function(s).

Adding a non-polar cosolvent promotes the formation of a heterogeneous medium and of two lipid phases, which compositions strongly differ. On one hand, lipid components unfunctionalized with one or more hydroxyl, epoxide, ketone, thiol, aldehyde, ether or amine function(s) will be found preferably in the non-polar phase, whereas the most polar lipid components, especially those functionalized with one or more hydroxyl, epoxide, ketone, thiol, aldehyde, ether and amine function(s), will be found preferably in the polar phase (light alcohol).

This step enables the selective extraction of non polar or weakly polar lipid components (unsaponifiable or not), which are not functionalized by any hydroxyl, epoxide, ketone, thiol, aldehyde, ether or amine function (or then at least few of them), which are separated from the lipid component mixture comprising one or more of these functions, preferably several of them (for example polyols), present in the medium following the transesterification reaction.

Depending on the type of raw material used, these lipid components that are not or only weakly polar may be, without limitation, fatty acid esters not containing any of hydroxyl, epoxide, ketone, thiol, aldehyde, ether and amine functions, furan lipids (in the case of avocado, furan lipid precursors have already been converted to furan lipids prior to beginning the reactive trituration step, these furan lipids being non hydroxylated), weakly polar alcohols, such as tocopherols, squalene, xanthophylls and esterified sterols.

Step b) is conducted under temperature, stirring and duration conditions sufficient to enable the extraction of triglycerides and other lipid components from the raw material and the transesterification of said triglycerides, leading to the formation of a mixture comprising especially fatty acid esters, glycerol, the native unsaponifiable fraction (unmodified by this step) and an oil cake. This step b), as opposed to that of the first method, is conducted without limitation as regards temperature, that is to say it may exceed 75 or 80° C. in every instance. Step b) is generally conducted at room temperature, but may also be conducted by implementing a heating process at a temperature ranging from 40 to 100° C.

The non-polar cosolvent, immiscible with the light alcohol (in the conditions of the reactive trituration), is preferably chosen so that lipid components, functionalized with one or more hydroxyl, epoxide, ketone, thiol, aldehyde, ether or amine function(s) and to be not extracted, are not soluble in such cosolvent. Considering their chemical nature, these functionalized lipid components will have necessarily a stronger affinity with the light alcohol phase than with the non-polar solvent phase in which they are not much (preferably not) soluble.

The reactive trituration step enables to recover (especially after filtration and washing of the oil cake with a solvent, such as a light alcohol), on one hand, two immiscible, liquid lipid phases, glycerol and, on the other hand, a solvent-containing oil cake. The polar phase (alcoholic phase, noted A), in which especially lipids functionalized by hydroxyl (preferably aliphatic) and/or epoxide groups, such as polyhydroxylated fatty alcohols, are soluble, is separated from the non-polar phase. Said non-polar phase further contains a particularly high proportion of fatty acid esters. The separation of the various fractions may occur in different ways, especially by centrifugation, decantation and/or distillation.

Thus the polar solvent phase may be submitted to a solvent evaporation step conducted under vacuum and at a suitable temperature. The vaporized solvent is then condensed for being recycled. The polar phase (phase A), once separated from the glycerol by decantation (followed with a washing operation with water, or not), primarily formed of alkylesters and unsaponifiable (or not) polar compounds may then be engaged in the molecular distillation so as to obtain on one hand, purified esters (in the distillate) and on the other hand, a distillation residue enriched with minor polar compounds. The extraction of these essentially unsaponifiable compounds is conducted according to methods that are known from the person skilled in the art. For example by conducting the following sequence: 1) saponification of the alkylesters, 2) liquid-liquid extraction enabling the separation of the unsaponifiable compounds from the soaps, 3) desolventizing of the solvent phase enriched with unsaponifiable matters and 4) final purification of the unsaponifiable matter. A further alternative consists in saponifying directly phase A and in extracting the essentially polar unsaponifiable compounds by 1) a liquid-liquid extraction enabling to separate the unsaponifiable compounds from the soaps, 2) a desolventizing of the solvent phase enriched with unsaponifiable matters and 3) a final purification of the unsaponifiable matter.

The non-polar cosolvent is evaporated from the non-polar phase enriched with lipids not containing any of the hydroxyl, epoxide, ketone, thiol, aldehyde, ether and amine functions (or few of them) (unsaponifiable or not) especially under reduced pressure. The lipid product obtained may be submitted to a step of neutralization (before or after the evaporation of the non-polar cosolvent, preferably before), preferably through an acid, then to a step of decantation or centrifugation, which enables to recover the residual glycerol on one hand and a lipid phase on the other hand, and/or to a step of filtration. The remaining lipid phase may then be washed with water and dried under vacuum.

The resulting lipid phase (phase typically containing alkylesters and enriched with non-polar, unsaponifiable (or not) compounds) is then submitted to a step c) of concentration to obtain a mixture enriched with an unsaponifiable fraction. As for the first method, the preferred concentration method is the molecular distillation.

Distillation generally enables to obtain a light fraction (first distillate), comprising high-purity esters (typically alkyl esters) of fatty acids, and at least one heavier fraction (second distillate or residue), comprising the unsaponifiable fraction diluted in esters (typically alkyl esters) of fatty acids, present in a non negligible amount.

In the case of avocado, the concentrate enriched with the unsaponifiable fraction (and depleted in fatty acid esters) contains at this stage furan lipids (generally representing around 10-15% by weight), which are less volatile than the fatty acid monoesters. These furanic compounds are only present as traces in the light fraction comprising essentially fatty acid esters.

The concentrate is then optionally submitted to steps d) of saponification of the mixture enriched with the unsaponifiable fraction and e) of extraction of the unsaponifiable fraction from the saponified mixture. Once extracted, the unsaponifiable fraction is preferably purified, using the same methods as those described for the first method of the invention.

The second method according to this invention enables to obtain a very pure unsaponifiable fraction, enriched with weakly polar or non-polar compounds. In a non-exhaustive manner, the unsaponifiable compounds obtained at the end of the implementation of such method in the fraction isolated in fine may be, depending on the nature of the raw material used, furan lipids (in the case of avocado), sterol esters, esterified triterpene alcohols, cholesterol esters, tocopherols (and corresponding tocotrienols), sesamolin, sesamin, sterenes, squalene, paraffin hydrocarbons, weakly to non-polar terpenes (mono-, di- and sesqui-terpenes with an aldehyde and/or a ketone function), esterified xanthophylls (lutein, astaxanthin, zeaxanthin), carotenoid type pigments (beta-carotene, lycopene), waxes, calciferol, cholecalciferol, pongamol.

In a general way, the average composition of an avocado unsaponifiable obtained following these different steps (amongst which steps d) and e)), as expressed in percentages by weight compared to the unsaponifiable total weight, is given thereunder:
furan lipids 60-80%
squalene 1-7%
others 5-20% (hydrocarbons, tocopherols, fatty ketones, heavy pigments . . . )
polyhydroxylated fatty alcohols 0.1-10%.

According to the present invention, the unsaponifiable matter obtained as described may then be submitted to a (second) step of distillation, so as to further improve the purity thereof, preferably a molecular distillation, conducted preferably at a temperature ranging from 100 to 160° C., more preferably from 100 to 140° C., under a pressure ranging preferably from 10-3 to 5.10-2 mm Hg. This (second) distillation may enable to obtain a distillate comprising primarily, in the case of avocado, avocado furan lipids, the purity of which may be higher than 90% by weight.

This second method of the invention thus enables to obtain a selective extraction of avocado furan lipids, except the polyhydroxylated fatty alcohols from avocado which have been extracted in the polar phase during the reactive trituration step.

Furthermore, the unsaponifiable compounds obtained at the end of the implementation of such method in the fraction isolated from the polar solvent phase, in fine may be, depending on the nature of the raw material used, the optionally polyhydroxylated fatty alcohols, furan lipids (in the case of avocado), sterols and non-esterified (free) or non-glycosylated triterpene alcohols, free and glycosylated polyphenols, free or sulfated cholesterol, lignanes, phorbol esters, triterpene acids (for ex. ursolic acid), polar terpenes (mono-, di- and sesqui-terpenes, with an alcohol function), alkaloids, polycosanols, limonoids, xanthophylls (lutein, astaxanthin, zeaxanthin) in a free form, gossypol, karanjin, shizandrin, azadirachtin, co-enzyme Q10, aflatoxins, especially B1 and B2, isoflavones, caffeine, theobromine, yohimbine, sylimarin, lupeol, althetoin.

The present invention has many advantages as compared to traditional existing methods used for the extraction from oils or deodorization emissions. First of all, the method of the invention is economical because it does not require the substantial investments of the traditional methods. As regards investment, the method of the invention enables to avoid the mechanical trituration tools like a screw press or a hexane extractor, and refining tools (mucilage removal, neutralization). Moreover, as opposed to mechanical trituration or evaporative trituration with hexane, and to refining, the reactive trituration according to the invention does not imply a high energy consumption. Moreover it requires a lower fresh water consumption than the refining operations of crude oils.

In addition, the present invention is very interesting as regards co-utilization, because implementing the methods of the invention leads to high-added value co-products, such as:
esters, generally alkylesters, of high purity, directly utilizable in cosmetics or in pharmacy (as opposed to methods of the prior art, which require a step of distillation for a triglyceride-containing mixture, such a distillation generating a strongly colored oil, difficult to purify because requiring a higher temperature than the one optionally used in the invention, which relates to a mixture containing fatty acid monoesters derived from the transesterification, lighter than triglycerides),
glycerol, which has many applications in cosmetics, pharmacy, hygiene, anti-gel fluids, etc.,
oil cakes, from which toxic or antinutritional compounds optionally present in the initial biomass have been removed, and which are directly utilizable in animal feeding or human nutrition, or oil cakes, sources of interesting oligopeptides and/or oligosaccharides,
polysaccharides and polyphenols utilizable in cosmetics, pharmacy and animal feeding and human nutrition.

From an economic and environmental point of view, the methods of the invention not only enable to reuse almost 100% of the fruit, as opposed to current methods and therefore to save biomass, or even cultivated areas, but they also enable to improve the whole value chain, from the farmer upstream to the user downstream, of said unsaponifiable matters. Lastly, they respect the key-principles of today's biorefinary models that are being developed for many applications, in particular for energetic and industrial purposes.

The unsaponifiable fractions obtained by the methods of the invention share a composition close or even similar to that of the unsaponifiable present in the raw material before the treatment.

Advantageously, these unsaponifiable fractions and these co-products of the invention are devoid of any residual toxic solvent and thus have a much better regulatory safety and acceptability as compared with products resulting from traditional methods. These particular characteristics enable a more adapted use of the unsaponifiable fractions obtained by the methods the invention and/or of the co-products provided, in cosmetic, drug, food compositions or food supplements or additives for humans and/or animals.

Likewise, the method of the invention will enable to separate and/or concentrate, depending on their polarity, the contaminants that may be present in vegetable or animal biomasses: polycyclic aromatic hydrocarbons (PAHs), pesticides, polychlorobiphenyls (PCB), dioxins, brominated flame retardants, pharmaceuticals, etc.

The avocado unsaponifiable fraction obtained by the methods of the invention may especially be used for preparing a drug for the treatment, for example, of joint affections, more particularly the treatment of osteoarthritis and for the treatment of arthritis (that is to say rheumatoid arthritis, psoriatic arthritis, Lyme disease and/or any other type of arthritis). The thus prepared drug may be intended for the treatment of periodontal diseases, and in particular for the treatment of periodontitis. This drug may furthermore be for treating osteoporosis. Moreover, this drug may be intended to modulate the nervous cell differentiation induced by NGF (Nerve Growth Factor). Lastly, this drug may be intended to repair tissues, and in particular the skin tissues, especially in the frame of a dermatological application.

The avocado unsaponifiable fraction derived from the methods of the invention may also be employed in cosmetic compositions, especially in dermocosmetics, for the cosmetic treatment of skin, adjacent mucosae and/or keratinized skin appendages (aging, scars . . . ), of capillar fibers or dermal papillae, in the presence of an excipient and/or a cosmetically acceptable vehicle.

Likewise, the co-products of the method such as proteins and carbon hydrates may, depending on their nature, lead as such or post transformation, to the production of active principles or excipients for use in pharmacy, cosmetics and human nutrition or animal feeding applications.

### EXAMPLES

### Selective Extraction of Unsaponifiable Compounds from Avocado

40 kg of whole Haas avocados are cut (stone included) in 0.5 cm-thick slices maximum. 20 kg of such slices are then dried in a ventilated drying oven at 70° C. for 16 hours (batch A). As a result, 1254 g of dried avocado are obtained after drying.

The 20 other kg are dried at 90° C. for 16 hours, so as to reach a residual moisture of 2% (batch B). As a result, 1327 g of dried avocado are obtained after drying.

The amounts of lipids in the homogenates A and B are then determined according to a standardized method (NF EN ISO 659).

Batch a is then Submitted to Following Actions:

1) coarse powder grinding (particle size ranging from 0.3 to 0.8 cm diameter).

2) introduction of the homogenate into a packed-bed percolation column (1100 g);

3) a biphasic solvent mixture ethanol (1100 g)/hexane (1100 g) and 3.6 g of caustic soda flakes as a catalyst (soda previously dissolved in ethanol) is then sent to the flake bed for 30 minutes at 40° C.

4) the biphasic miscella (solvent phase resulting from the liquid-solid extraction) is then racked off. The flake bed is then washed through 5 successive washing operations with the ethanol/hexane mixture at 40° C. (5 minutes per washing).

5) the biphasic miscella is then centrifuged so as to separate ethanol and hexane phases. The solvents of both recovered organic phases are then evaporated under a 20 mbar vacuum, at 90° C. for 20 minutes. The glycerol is then separated from the lipid phase ex-ethanol by a simple centrifugation.

6) lipids obtained after evaporation of their respective solvent (ethanol or hexane), together with, or not, insoluble gums (oil-insoluble products extracted from the flake during the process), are washed to neutrality by adding hot water and centrifuging. Lastly, they are dried under a 20 mbar vacuum, at 90° C., for 5 minutes. As a result, respectively 412 g of lipids derived from the hexane phase and 176 g derived from the ethanol phase are obtained.

The lipids of the hexane phase are then analyzed as follows:
saponification number (method NF ISO3657): 186.4 mg KOH/g
acid value (method NFT 60-204): 2.1 mg KOH/g
unsaponifiable content (method NF ISO 3596 modified, with dichloroethane as extraction solvent): 0.32%.

A thin-layer chromatography analysis indicates that the lipids only contain a few traces of avocado polyhydroxylated fatty alcohols and of furan precursors.

As a consequence, the method indeed leads to the formation of a non-polar lipid phase depleted in avocado unsaponifiable polar compounds.

The lipids derived from the ethanol phase are then heated in a flask provided with a Dean-stark at 120° C. for 12 hours. After this heat treatment, the thin-layer chromatography (TLC) analysis indicates that the lipids comprise avocado furanic compounds and polyhydroxylated fatty alcohols in a high amount, these compounds having TLC specific spots.

The lipids of the ethanol phase are analytically analyzed as follows:
saponification number (method NF ISO3657): 171.1 mg KOH/g
acid value (method NFT 60-204): 3.3 mg KOH/g
unsaponifiable content (method NF ISO 3596 modified, with dichloroethane as extraction solvent): 6.1%.

Considering the analysis characterizations, the implemented extraction method indeed enables to selectively extract the dried avocado unsaponifiable polar compounds (TLC spots specific for polyhydroxylated fatty alcohols and furan lipids derived from the cyclization of furan precursors resulting from the heat treatment), together with an outstanding yield (unsaponifiable content much higher than 4%).

The unsaponifiable fraction is then washed, dried, concentrated by molecular distillation, saponified, extracted and purified according to the same protocol as in Example n° 2 of the world application WO 2011/048339.

A TLC analysis of the unsaponifiable matter reveals spots specific for furan lipids (very strong spots), hydroxylated fatty alcohols (weaker spots) and phytosterols (weak spots).

Batch B is then Submitted to the Following Actions:

1) coarse powder grinding (particle size ranging from 0.3 to 0.8 cm diameter);

2) introduction of the homogenate into the packed-bed percolation column;

3) a solvent biphasic mixture of ethanol (1100 g)/hexane (1100 g) and 3.6 g of caustic soda flakes as a catalyst (soda previously dissolved in ethanol) is then sent to the flake bed for 30 minutes at 40° C.;

4) the biphasic miscella (solvent phase resulting from the liquid-solid extraction) is then racked off. The flake bed is then washed through 5 successive washing operations with the ethanol/hexane mixture at 40° C. (5 minutes per washing);

5) the biphasic miscella is then centrifuged so as to separate ethanol and hexane phases. Both recovered organic phases are then evaporated under a 20 mbar vacuum, at 90° C. for 20 minutes. The glycerol is then separated from the ex-ethanol lipid phase by a simple centrifugation;

6) lipids obtained after evaporation of their respective solvent (ethanol or hexane), together with, or not, insoluble gums (oil-insoluble products extracted from the flake during the process), are washed to neutrality by adding hot water and centrifuging. Lastly, they are dried under a 20 mbar vacuum, at 90° C., for 5 minutes. As a result, respectively 412 g of lipids derived from the hexane phase and 176 g derived from the ethanol phase are obtained.

The lipids of the hexane phase are then analyzed as follows:
saponification number (method NF ISO3657): 179.5 mg KOH/g
acid value (method NFT 60-204): 0.9 mg KOH/g
unsaponifiable content (method NF ISO 3596 modified, with dichloroethane as extraction solvent): 5.3%.

A thin-layer chromatography analysis indicates that the lipids of the hexane phase are primarily formed of furan lipids with some traces of polyhydroxylated fatty alcohols.

As a consequence, the method indeed leads to the formation of a non-polar lipid phase enriched with furan lipids and depleted in unsaponifiable polar compounds of avocado such as polyhydroxylated fatty alcohols.

The lipids derived from the ethanol phase are analyzed by a thin-layer chromatography (TLC). Such analysis indicates that this phase contains avocado polyhydroxylated fatty alcohols in a high amount, as well as furan lipids as traces.

The lipids of the ethanol phase are analytically characterized as follows:
saponification number (method NF ISO3657): 176.1 mg KOH/g
acid value (method NFT 60-204): 3.1 mg KOH/g
unsaponifiable content (method NF ISO 3596, modified, with dichloroethane as extraction solvent): 1.2%.

Considering the analysis characterizations, the implemented extraction indeed enables to selectively extract the polar compounds from dried avocado unsaponifiable (TLC spots specific for polyhydroxylated fatty alcohols).

The unsaponifiable fraction of the lipids derived from the hexane phase is then washed, dried, concentrated by molecular distillation, saponified, extracted and purified according to the same protocol as in Example n° 2 of the application WO 2011/048339.

A TLC analysis of the unsaponifiable matter obtained reveals specific spots for furan lipids (very strong spots), for hydroxylated fatty alcohols (very weak spots) and for phytosterols (very weak spots).

The solvent-containing oil cakes derived from the transformation of batches A and B of dried fruits are then desolvented in a drying oven at 70° C. for 16 hours. Their respective contents in lipids, as determined using the method described in Standard NF EN ISO 659 are as follows:
oil cake batch A: 0.7%/dry matter
oil cake batch B: 0.6%/dry matter

As a consequence, the method leads to the formation of delipidated, and thus protein- and polysaccharide enriched oil cakes, which are sources of active principles and/or excipients, or that may also be used, as such, in human nutrition and animal feeding applications.

1-11. (canceled)

12. A method for extracting an unsaponifiable fraction from a renewable raw material comprising lipids functionalized with one or more function(s) chosen from hydroxyl, epoxide, ketone, thiol, aldehyde, ether and amine functions, comprising the following steps:
a) dehydration optionally preceded or followed with a conditioning of the renewable raw material,
b) reactive trituration of the raw material dehydrated and optionally conditioned, in the presence of at least one polar organic solvent comprising at least one light alcohol, at least one non-polar cosolvent immiscible with said light alcohol and at least one catalyst, resulting in the formation of a polar organic phase enriched with lipids functionalized with one or more function(s) chosen from hydroxyl, epoxide, ketone, thiol, aldehyde, ether and amine functions,
c) concentration of the polar organic phase to obtain a mixture enriched with an unsaponifiable fraction, optionally preceded, accompanied or followed with a heat treatment at a temperature higher than or equal to 75° C., preferably higher than or equal to 80° C.,
and comprising optionally the following steps:
d) saponification of the mixture enriched with the unsaponifiable fraction,
e) extraction of the unsaponifiable fraction from the saponified mixture.

13. A method for extracting an unsaponifiable fraction from a renewable raw material comprising the following steps:
a) dehydration optionally preceded or followed with a conditioning of the renewable raw material,
b) reactive trituration of the raw material dehydrated and optionally conditioned, in the presence of at least one polar organic solvent comprising at least one light alcohol, at least one non-polar cosolvent immiscible with said light alcohol and at least one catalyst, resulting in the formation of a non-polar organic phase enriched with lipids containing no or few hydroxyl, epoxide, ketone, thiol, aldehyde, ether and amine function(s),
c) concentration of the non-polar organic phase to obtain a mixture enriched with an unsaponifiable fraction,
and comprising optionally the following steps:
d) saponification of the mixture enriched with the unsaponifiable fraction,
e) extraction of the unsaponifiable fraction from the saponified mixture,
wherein said renewable raw material undergoes optionally a heat treatment at a temperature higher than or equal to 75° C., preferably higher than or equal to 80° C., before or during step b).

14. The method according to claim 13, wherein said heat treatment is carried out, and concomitantly to step a) of dehydration.

15. The method according to claim 12, wherein the renewable raw material is chosen from the fruit, the stone, the leaves of avocado and their mixtures, said heat treatment is carried out, and steps a) and b) are conducted at a temperature lower than or equal to 80° C., preferably lower than or equal to 75° C.

16. The method according to claim 13, wherein the renewable raw material is chosen from the fruit, the stone, the leaves of avocado and their mixtures, and said heat treatment is carried out.

17. The method according to claim 12, wherein the dehydration is conducted so as to reach a residual moisture lower than or equal to 3% by weight, as compared to the weight of the raw material obtained at the end of the dehydration step.

18. The method according to claim 12, wherein the light alcohol is chosen from methanol, ethanol, propanol, isopropanol, butanol, pentanol, hexanol, ethyl-2-hexanol, and isomers thereof.

19. The method according to claim 12, wherein the non-polar cosolvent is an alkane or a mixture of alkanes.

20. The method according to claim 12, wherein the catalyst is a basic catalyst.

21. The method according to claim 12, wherein the concentration of the organic phase is effected by molecular distillation.

22. The method according to claim 12, further defined as comprising steps d) and e), the extraction of the unsaponifiable fraction from the saponified mixture being carried out by liquid-liquid extraction using at least one organic solvent.

23. The method according to claim 13, wherein the dehydration is conducted so as to reach a residual moisture lower than or equal to 3% by weight, as compared to the weight of the raw material obtained at the end of the dehydration step.

24. The method according to claim 13, wherein the light alcohol is chosen from methanol, ethanol, propanol, isopropanol, butanol, pentanol, hexanol, ethyl-2-hexanol, and isomers thereof.

25. The method according to claim 13, wherein the non-polar cosolvent is an alkane or a mixture of alkanes.

26. The method according to claim 13, wherein the catalyst is a basic catalyst.

27. The method according to claim 13, wherein the concentration of the organic phase is effected by molecular distillation.

28. The method according to claim 13, further defined as comprising steps d) and e), the extraction of the unsaponifiable fraction from the saponified mixture being carried out by liquid-liquid extraction using at least one organic solvent.
